(19) United States
(12) Patent Application Publication
(10) Pub. No.: US 2020/0069882 A1
(43) Pub. Date: Mar. 5, 2020

(54) DOSE RECORDING DEVICE AND DOSE RECORDING METHOD

(71) Applicant: ALTEK BIOTECHNOLOGY CORPORATION, Hsinchu City (TW)

(72) Inventor: Hsi-Hsin Loo, Taipei City (TW)

(73) Assignee: ALTEK BIOTECHNOLOGY CORPORATION, Hsinchu City (TW)

(21) Appl. No.: 16/368,898

(22) Filed: Mar. 29, 2019

(30) Foreign Application Priority Data
Aug. 31, 2018 (TW) 107130668

(51) Int. Cl. A61M 5/315

(52) U.S. Cl. CPC A61M 5/31525; A61M 2005/3126; A61M 5/3155

(57) ABSTRACT

A dose recording device adapted to be detachably sleeved on an injection pen having a wheel, a dial gauge and a press button both linked with the wheel is provided. The dose recording device includes a casing, a circuit board module, at least one optical sensor, a motion sensor and an adjustable fixing assembly. The casing includes a through hole for the injection pen passing through. The optical sensor is electrically connected to the circuit board module for detecting at least one of a value on the dial gauge and a position of the press button. The motion sensor is electrically connected to the circuit board module for detecting rotating vibration of the wheel and inversion state of the injection pen. The adjustable fixing assembly is for fixing the casing to the injection pen. A dose recording method is further provided.

### CROSS-REFERENCE TO RELATED APPLICATION

This application claims the priority benefit of Taiwan application serial no. 107130668, filed on Aug. 31, 2018. The entirety of the above-mentioned patent application is hereby incorporated by reference herein and made a part of this specification.

### BACKGROUND OF THE INVENTION

### Field of the Invention

The disclosure relates to a dose recording device and a dose recording method, and particularly relates to a dose recording device and a dose recording method adapted for an injection pen.

### Description of Related Art

For injection pens currently on the market (such as insulin injection pens), whether it is a disposable injection pen or a dedicated injection pen with a replaceable medicine cartridge, the injection dose is generally controlled by rotating the wheel. When the wheel on the injection pen is rotated, the dial gauge on the injection pen is linked with the wheel, so as for the user to easily see the value of the dose to be injected into the body. Besides, the press button on the injection pen is also linked with the wheel and gradually increases the pressing path as the wheel rotates, so as for the user to subsequently executes the injection procedure by pressing the press button. At present, the dose and time of each injection are mostly dependent on the patient's self-recording. If any insufficient or excessive injection is caused by omission or error, the patient may be at health risk.

### SUMMARY OF THE INVENTION

A dose recording device adapted to be detachably sleeved on an injection pen and to record an injection information of the injection pen is provided.

A dose recording method adapted for recording an injection information of an injection pen accurately is also provided.

A dose recording device of the disclosure is adapted to be detachably sleeved on an injection pen. The injection pen includes a wheel, a dial gauge and a press button both linked with the wheel. The dose recording device includes a casing, a circuit board module, at least one optical sensor, a motion sensor and an adjustable fixing assembly. The casing includes a through hole for the injection pen passing through. The circuit board module is disposed at the casing. At least one optical sensor is disposed at the casing and electrically connected to the circuit board module. The at least one optical sensor is adapted for detecting at least one of a value on the dial gauge and a position of the press button. The motion sensor is disposed at the casing and electrically connected to the circuit board module. The motion sensor is adapted for detecting a vibration of the wheel as the wheel rotates and an inversion state of the injection pen. The adjustable fixing assembly is disposed at the casing and is adapted for fixing the casing to the injection pen.

In an embodiment of the disclosure, the at least one optical sensor includes two optical sensors, one of the two optical sensors is disposed at a part of the casing corresponding to the dial gauge and is adapted to sense the value on the dial gauge, and the other one of the two optical sensors is disposed at a part of the casing corresponding to the press button and is adapted for sensing the position of the press button.

In an embodiment of the disclosure, the adjustable fixing assembly includes a rotary knob rotatably disposed at the casing. The rotary knob is adapted to adjust an inner diameter of the adjustable fixing assembly for fixing the casing to the injection pen.

In an embodiment of the disclosure, the dose recording device further includes a display screen exposed from the casing and electrically connected to the circuit board module.

In an embodiment of the disclosure, the dose recording device further includes a wireless communication module disposed at the casing and electrically connected to the circuit board module.

A dose recording method of the disclosure is adapted for recording an injection information of an injection pen. The injection pen includes a liquid outlet end, a wheel, a dial gauge linked with the wheel and a press button linked with the wheel. The dose recording method includes the following. An optical sensing process is executed, wherein the optical sensing process includes sensing at least one of a position of the press button of the injection pen and a value on the dial gauge of the injection pen. A vibration sensing process is executed, wherein the vibration sensing process includes sensing the vibration of the wheel of the injection pen as the wheel rotates. The information of the dose intended to be output by the injection pen is determined according to both the information sensed via the optical sensing process and the information sensed via the vibration sensing process. When the press button is being pressed, a gravity sensing process is executed to determine whether the injection pen is in a work mode or in an air clearing mode. In the work mode, the liquid outlet end of the injection pen is at a lower position in the gravity direction with respect to the wheel, and in the air clearing mode, the liquid outlet end of the injection pen is at an upper position in the gravity direction with respect to the wheel. The injection information of the injection pen in the work mode is recorded, wherein the injection information includes a value of change of the dial gauge and an injection time.

In an embodiment of the disclosure, a user's dose confirmation process is executed before the injection pen executes the work mode, wherein the information of the dose intended to be output by the injection pen is output for the user's confirmation.

In an embodiment of the disclosure, the injection information is output after the injection pen executes the work mode.

In an embodiment of the disclosure, the optical sensing process includes sensing a position of the press button of the injection pen and a value on the dial gauge of the injection pen.

In an embodiment of the disclosure, the dose recording method further include recording a time information of the injection pen in the air clearing mode.

Based on the above, the dose recording device of the disclosure is adapted to be detachably sleeved on an injection pen and has an adjustable fixing assembly for fixing the casing to the injection pen, such that the dose recording device of the disclosure is applicable to injection pens of different diameters and models. In addition, the dose recording device of the disclosure is adapted for applying the dose recording method of the disclosure to record the injection information of the injection pen. More specifically, the dose recording device of the disclosure detects at least one of the value on the dial gauge of the injection pen and the position of the press button of the injection pen by the optical sensor and detects the vibration of the wheel of the injection pen as the wheel rotates by the motion sensor. As such, the dose to be injected by the injection pen is double confirmed so as to reduce the chances of misjudging the dose to be injected by the injection pen. In addition, since the motion sensor of the disclosure is adapted to detect the inversion state of the injection pen, the dose recording device may identify whether the injection pen is in the working mode or in the air clearing mode according to the detection result by the motion sensor, so as to avoid mistakenly recording the amount of air clearing as the injection dose.

To make the above features and advantages of the disclosure more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a schematic view of a dose recording device sleeved on an injection pen according to an embodiment of the disclosure.

FIG. 2 is a schematic view of the dose recording device of FIG. 1 separated from the injection pen.

FIG. 3 and FIG. 4 are respectively schematic views of the interior of the dose recording device of FIG. 1 viewed in different angles.

FIG. 5 is a flowchart of a dose recording method according to an embodiment of the disclosure.

### DESCRIPTION OF THE EMBODIMENTS

Some other embodiments of the invention are provided as follows. It should be noted that the reference numerals and part of the contents of the previous embodiment are used in the following embodiments, in which identical reference numerals indicate identical or similar components, and repeated description of the same technical contents is omitted. Please refer to the description of the previous embodiment for the omitted contents, which will not be repeated hereinafter.

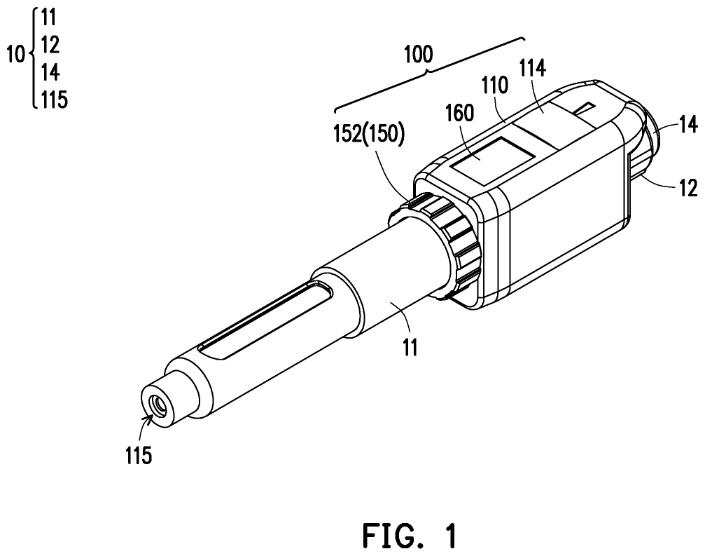

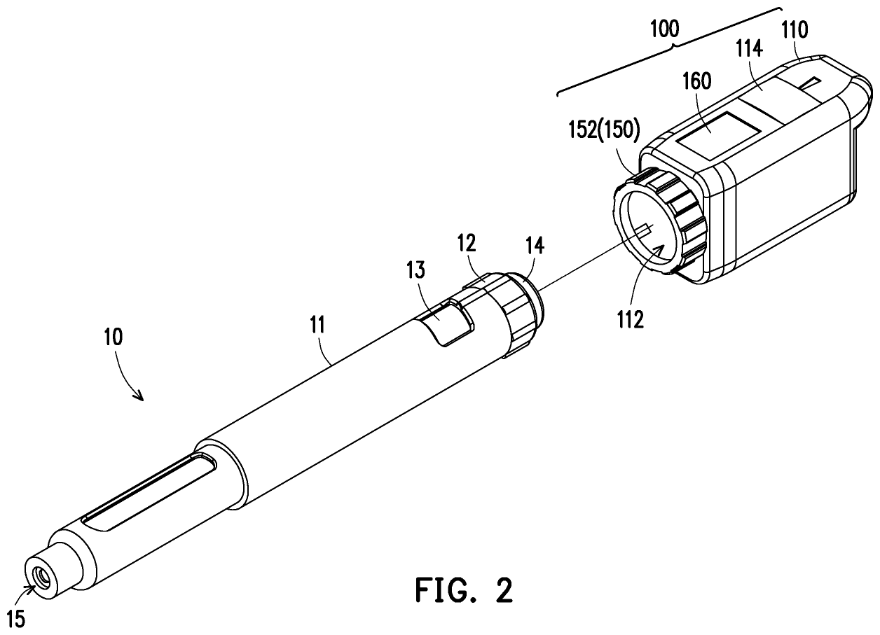

FIG. 1 is a schematic view of a dose recording device sleeved on an injection pen according to an embodiment of the disclosure. FIG. 2 is a schematic view of the dose recording device of FIG. 1 separated from the injection pen. Referring to FIG. 1 to FIG. 2, In this embodiment, the injection pen 10 is, for example, an injection pen of insulin, and the injection pen 10 may be a disposable injection pen or a dedicated injection pen with a replaceable medicine cartridge. The type of the injection pen 10 is not limited thereto. As shown in FIG. 2, the injection pen 10 includes a body 11, a liquid outlet end 15 located at one end of the body 11, a wheel 12 that is rotatable relative to the body 11 and is near the other end of the body 11, and a dial gauge 13 and a press button 14. The dial gauge 13 and the press button 14 are both linked with the wheel 12. The injection pen 10 may have a needle (not shown) at the liquid outlet end 15, and the injection liquid (for example, insulin) is adapted to be injected into the human body through the liquid outlet end 15 and the needle.

The injection pen 10 generally controls the injected dose by rotating the wheel 12. When the wheel 12 on the injection pen 10 rotates, the dial gauge 13 on the injection pen 10 is linked with the wheel 12, so as for the user to easily see the value of the dose to be injected into the body. Also, the press button 14 on the injection pen 10 is linked with the wheel 12 and gradually moves away from the liquid outlet end 15 as the wheel 12 rotates, providing a path for the press button 14 to be pressed subsequently. In general, as the angle or number of rotations of the wheel 12 increases, the distance that the press button 14 moves away from the liquid outlet end 15 increases, and the pressing path of the press button 14 also increases. After rotating the wheel 12 to a fixed position, the user simply presses the press button, and the injection liquid is squeezed out from the liquid outlet end 15 to execute the injection procedure. In addition, when beginning to use the injection pen 10, since there is air in the needle, the user needs to rotate the wheel 12 by a small angle, then turn the liquid outlet end 15 of the injection pen 10 upward (that is, turning the needle facing up) and press the press button 14, so as to achieve the effect of air clearing. This procedure is to avoid injecting air into the human body and to avoid mistake on the dose to be injected.

At present, the dose and time of each injection are mostly dependent on the user's self-recording, and it is very likely that the user may forget to record and thus repeat unnecessary injections or skip one injection. The dose recording device 100 of this embodiment is installable on the injection pen 10 of different sizes and models, and automatically records the injection information of the injection pen 10 (for example, the time and dose of the injection), which may effectively solve the above problems. As such, the user may know the time and dose of the previous injection by checking the information in the dose recording device 100, and thereby know the time and dose of the next injection. In addition, the dose recording device 100 of this embodiment ensures the correctness of the injected dose through a dual mechanism of optical sensing and motion sensing. Besides, the dose recording device 100 of this embodiment may also sense the inversion state of the injection pen 10 to distinguish whether the press button 14 is pressed for air clearing or for injecting. The dose recording device 100 and the operation method thereof are to be described below.

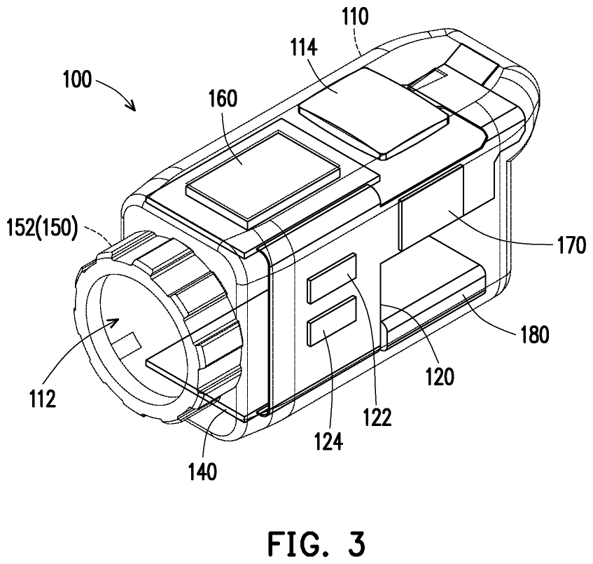

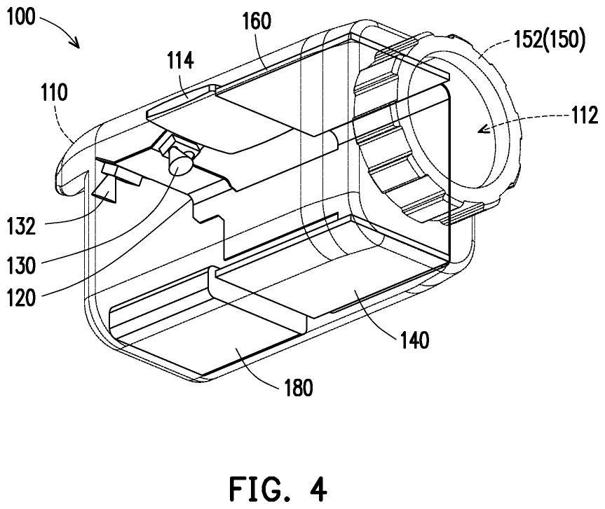

FIG. 3 and FIG. 4 are respectively schematic views of the interior of the dose recording device of FIG. 1 viewed in different angles. In FIG. 3 and FIG. 4, in order to clearly show the internal components of the dose recording device 100, the casing 110 is indicated by a broken line. Referring to FIG. 3 to FIG. 4, the dose recording device 100 of this embodiment includes a casing 110, a circuit board module 120, at least one optical sensor 130 and 132, a motion sensor 140 and an adjustable fixing assembly 150. The casing 110 includes a through hole 112 adapted for the injection pen 10 passing through. In addition, in this embodiment, the casing 110 further includes a transparent window 114, and the position of the transparent window 114 corresponds to the position of the dial gauge 13 (as indicated in FIG. 2) of the injection pen 10, so that the user can easily see the value on the dial gauge 13. Of course, in other embodiments, the transparent window 114 may also be omitted.

The circuit board module 120 is disposed at the casing 110. In this embodiment, the circuit board module 120 includes a flexible circuit board, a processor 122 and a storage 124. The processor 122 and the storage 124 are disposed at and electrically connected to the flexible circuit board. However, the type and form of the circuit board module 120 are not limited thereto.

As shown in FIG. 4, the optical sensors 130 and 132 are disposed at the casing 110 and electrically connected to the circuit board module 120. The optical sensors 130 and 132 are adapted to detect at least one of the value on the dial gauge and the position of the press button 14. As shown in FIG. 4, in this embodiment, a number of the optical sensors 130 and 132 is two optical sensors 130, 132, wherein one optical sensor 130 is disposed at a part of the casing 110 corresponding to the dial gauge of the injection pen 10 and is adapted to sense the value on the dial gauge, and the other optical sensor 132 is disposed at a part of the casing 110 corresponding to the press button 14 of the injection pen 10 and is adapted to sense the position of the press button 14. More specifically, since the press button 14 moves away from the liquid outlet end 15 of the body 11 after the wheel 12 rotates, the optical sensor 132 may sense the position of the press button 14 relative to the body 11 so as to determine whether the injection of the injection pen 10 is completed or not. In addition, in this embodiment, the optical sensors 130 and 132 are, for example, charge-coupled devices (CCD) or infrared sensors. In other embodiments, the dose recording device 100 may also have only one single optical sensor for detecting the value on the dial gauge 13 or detecting the position of the press button 14. The number, type and dispositional position of the optical sensors 130 and 132 are not limited thereto.

The motion sensor 140 is disposed at the casing 110 and electrically connected to the circuit board module 120. The motion sensor 140 is adapted for detecting the vibration of the wheel 12 as the wheel 12 rotates and the inversion state of the injection pen 10. In this embodiment, the motion sensor 140 is, for example, a gravity sensor (G sensor), but the type of the motion sensor 140 is not limited thereto. In general, in order to provide the user with good feeling in hand, the elastic structure inside the wheel 12 generates a vibration feedback and a sense of sections when the wheel 12 of the injection pen 10 rotates. The motion sensor 140 of this embodiment may sense the vibration of the wheel 12 of the injection pen 10 as the wheel 12 rotates to double confirm how many scales of the wheel 12 the user has turned. In addition, the vibration of the wheel 12 when rotating forward is slightly different from that when rotating backward, such that the motion sensor 140 may also sense the forward rotation and the backward rotation of the wheel 12 and thereby avoid miscalculating the position, the angle of rotation or/and the number of rotations of the wheel 12.

In addition, the adjustable fixing assembly 150 is disposed at the casing 110 and is adapted for fixing the casing 110 to the injection pen 10. In this embodiment, the adjustable fixing assembly 150 includes a rotary knob 152 rotatably disposed at the casing 110. The rotary knob 152 is adapted to adjust an inner diameter of the adjustable fixing assembly 150 for fixing the casing 110 to the injection pen 10. In general, a diameter of the injection pen 10 varies with different brands thereof, but a diameter of the injection pen 10 is roughly 15 mm to 17 mm. In this embodiment, the inner diameter of the through hole 112 on the dose recording device 100 for the injection pen 10 to pass through may be, for example, between 17 mm and 19 mm so as to ensure that the injection pen 10 of different sizes or models may pass through. After the injection pen 10 inserts into the casing 110, the user may adjust the inner diameter of the adjustable fixing assembly 150 by turning the rotary knob 152 of the adjustable fixing assembly 150, such that the dose recording device 100 is applicable to the injection pen 10 on the market.

In other words, the inner diameter of the adjustable fixing assembly 150 may be adjusted to be about 15 mm to 17 mm for the adjustable fixing assembly 150 to abut against the surface of the injection pen 10, and thereby fixing the casing 110 to the injection pen 10. As such, the dose recording device 100 of this embodiment may be widely used on various injection pens 10 on the market without changing the patient's using habits. Of course, in other embodiments, the adjustable fixing assembly 150 may also be a clamp, a strap, or in other forms that allows the dose recording device 100 to be fixed on the injection pen 10. The type of the adjustable fixing assembly 150 is not limited to the above.

In this embodiment, the dose recording device 100 further includes a display screen 160 exposed from the casing 110 and electrically connected to the circuit board module 120. The information sensed by the optical sensors 130 and 132 and the motion sensor 140 may be output to the display screen 160 for the user to view after being processed by the processor 122. The processor 122 may display the indication data of at least one of the current time, dose, operation state, power state, etc. on the display screen 160.

In addition, in this embodiment, the dose recording device 100 further includes a wireless communication module 170 disposed at the casing 110 and electrically connected to the circuit board module 120. After being processed by the processor 122, the information may not only be displayed on the display screen 160 but may also be transmitted to an external device through the wireless communication module 170, such as a WIFI module or a Bluetooth module. For example, the information may be transmitted to the user's mobile phone, computer or cloud system to inform the user or/and other related person of the information of the injection.

In addition, in this embodiment, the dose recording device 100 further includes a battery 180 electrically connected to the circuit board module 120, the optical sensors 130 and 132, the motion sensor 140 and the display screen 160 for power supply. The battery 180 may be a rechargeable battery or a disposable battery, and the type of the battery 180 is not limited thereto. In addition, in an embodiment not shown, the dose recording device 100 may also have components such as a magnifying lens or an illumination component.

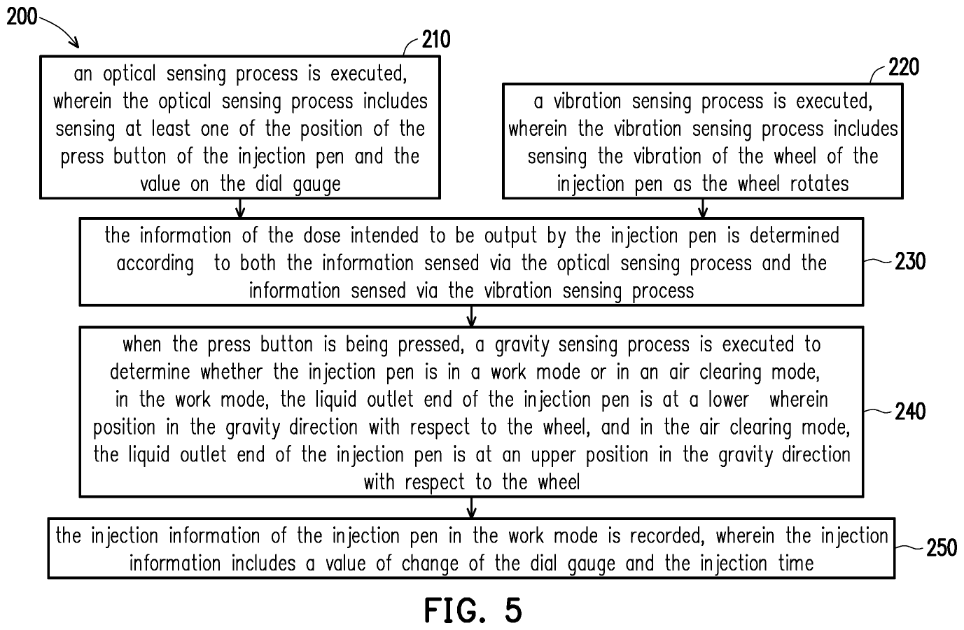

A dose recording method 200 is to be described below. It should be noted that, the dose recording method 200 described below may be applied to the above-described dose recording device 100, but it may also be applied to other devices as well. FIG. 5 is a flowchart of a dose recording method according to an embodiment of the disclosure. Referring to FIG. 1 to FIG. 5, the dose recording method 200 of this embodiment includes the following steps.

First, an optical sensing process is executed, wherein the optical sensing process includes sensing at least one of the position of the press button 14 of the injection pen 10 and the value on the dial gauge 13 (step 210). During the adjustment of the injection dose and during the injection, the value on the dial gauge 13 of the injection pen 10 or/and the position of the press button 14 may change. In this embodiment, the optical sensing process may include sensing both the position of the press button 14 of the injection pen 10 and the value on the dial gauge 13 so as to determine the dose intended to be output. Of course, in other embodiments, it is also possible to detect only one of the position of the press button 14 and the value on the dial gauge 13.

Additionally, a vibration sensing process is executed, wherein the vibration sensing process includes sensing the vibration of the wheel 12 of the injection pen 10 as the wheel 12 rotates (step 220). Since the elastic structure inside the wheel 12 also generates a vibration feedback and a sense of sections when the wheel 12 of the injection pen 10 rotates (for example, each turning of one scale makes a corresponding vibration), and even the vibration of the wheel 12 when rotating forward has a different vibration amplitude from that when rotating backward, the vibration sensing process may determine the dose intended to be output by sensing the vibration of the wheel 12 of the injection pen 10 as the wheel 12 rotates.

It should be noted that, in this embodiment, step 210 and step 220 have no specific sequence. Step 210 and step 220 may be executed simultaneously, or step 210 may be executed first, or step 220 may be executed first.

Next, the information of the dose intended to be output by the injection pen 10 is determined according to both the information sensed via the optical sensing process and the information sensed via the vibration sensing process (step 230). In other words, in the dose recording method 200 of this embodiment, the intended injection dose is not only sensed via an optical method but also assured via a vibration sensing method. The optical information and motion information are used together to determine the information of the dose intended to be output by the injection pen 10, so as to ensure that the dose information of the dose intended to be output is correct.

It is worth noting that, after step 230, the dose recording method 200 of this embodiment may execute a user's dose confirmation process optionally. For example, the value of the dose to be output by the injection pen 10 may be output for the user's confirmation, such as displaying the value of the dose to be output on the display screen 160 of the dose recording device 100 for the user's confirmation before injecting. As such, the dose of the injection may be ensured to be correct. Of course, the user's dose confirmation process may also be omitted.

Next, the user just needs to press the injection button 14 of the injection pen 10 to flow the injection liquid through the liquid outlet end 15 and the needle. In this embodiment, when the press button 14 is being pressed, a gravity sensing process is executed to determine whether the injection pen 10 is in a work mode or in an air clearing mode. In the work mode, the liquid outlet end 15 of the injection pen 10 is at a lower position in the gravity direction with respect to the wheel 12, and in the air clearing mode, the liquid outlet end 15 of the injection pen 10 is at an upper position in the gravity direction with respect to the wheel 12 (step 240).

The press button 14 of the injection pen 10 may be pressed for two reasons: either for clearing the air inside the needle or for injecting the injection liquid into the body. In the mode of clearing the air in the needle, the user presses the press button 14 to squeeze out the air after turning the needle facing up. On the other hand, when injecting the injection liquid into the body, the user generally moves the pen to the injection site of the body with the needle facing down, presses the press button 14 after the needle contacts the skin, and then stops for a few seconds for the medicine to be completely injected. In other words, the purpose of the current operation of the injection pen 10 is identifiable by determining the posture of the injection pen 10 (whether the liquid outlet end 15 of the injection pen 10 is facing up or down).

In addition, when the press button 14 is pressed, the change of the scale on the dial gauge 13, the change of the position of the press button 14 and the slight sectional vibration feedback caused by the rapid backward rotation of the wheel 12 may all be simultaneously detected by the optical sensors 130 and 132 and the motion sensor 140. In the work mode and the air clearing mode, the optical sensors 130 and 132 and the motion sensor 140 detect different changes, which may also serve as a basis for determining the user's motion. Therefore, by the above sensing processes, the posture, the inversion state and the operation state of the injection pen 10 may be determined so as to confirm the dose and purpose of the current operation, such that the air clearing process and the normal injection process may not be misidentified.

Next, the injection information of the injection pen 10 in the work mode is recorded. The injection information includes a value of change of the dial gauge (i.e. the injection dose) and the injection time (step 250). When the current injection is determined to be in the work mode, the processor 122 may record the injection information, that is, the injection dose and the injection time, in the storage 124. After step 250, the dose recording method 200 of this embodiment may optionally include outputting the injection information. The above injection information may be displayed on a display screen, such as the display screen 160 of the dose recording device 100, or on a display screen of an external device (for example, a mobile phone, a computer, etc.). Moreover, after step 240, the dose recording method 200 of this embodiment may also optionally include recording a time information when the injection pen 10 is in the air clearing mode. In other words, the user may also obtain the information of when a new injection pen 10 or a new injection tube was started to be used.

In sum of the above, the dose recording device of the disclosure is adapted to be detachably sleeved on an injection pen and has an adjustable fixing assembly for fixing the casing to the injection pen, such that the dose recording device of the disclosure is applicable to injection pens of different diameters and models. In addition, the dose recording device of the disclosure is adapted for applying the dose recording method of the disclosure to record the injection information of the injection pen. More specifically, the dose recording device of the disclosure detects at least one of the value on the dial gauge of the injection pen and the position of the press button of the injection pen by the optical sensor and detects the vibration of the wheel of the injection pen as the wheel rotates by the motion sensor. As such, the dose to be injected by the injection pen is double confirmed so as to reduce the chances of misjudging the dose to be injected by the injection pen. In addition, since the motion sensor of the disclosure is adapted to detect the inversion state of the injection pen, the dose recording device may identify whether the injection pen is in the working mode or in the air clearing mode according to the detection result by the motion sensor, so as to avoid mistakenly recording the amount of air clearing as the injection dose.

It will be apparent to those skilled in the art that various modifications and variations can be made to the disclosed embodiments without departing from the scope or spirit of this disclosure. In view of the foregoing, it is intended that the disclosure covers modifications and variations provided that they fall within the scope of the following claims and their equivalents.

What is claimed is:

1. A dose recording device, adapted for being detachably sleeved on an injection pen, the injection pen comprising a wheel, a dial gauge linked with the wheel, and a press button linked with the wheel, the dose recording device comprising:
a casing, comprising a through hole adapted for the injection pen passing through;
a circuit board module, disposed at the casing;
at least one optical sensor, disposed at the casing and electrically connected to the circuit board module, wherein the at least one optical sensor is adapted for detecting at least one of a value on the dial gauge and a position of the press button;
a motion sensor, disposed at the casing and electrically connected to the circuit board module, wherein the motion sensor is adapted for detecting a vibration as the wheel rotates and an inversion state of the injection pen; and
an adjustable fixing assembly, disposed at the casing and adapted for fixing the casing to the injection pen.

2. The dose recording device according to claim 1, wherein the at least one optical sensor comprises two optical sensors, one of the two optical sensors is disposed at a part of the casing corresponding to the dial gauge and is adapted for sensing the value on the dial gauge, and the other one of the two optical sensors is disposed at a part of the casing corresponding to the press button and is adapted for sensing the position of the press button.

3. The dose recording device according to claim 1, wherein the adjustable fixing assembly comprises a rotary knob rotatably disposed at the casing, the rotary knob is adapted for being rotated to adjust an inner diameter of the adjustable fixing assembly for fixing the casing to the injection pen.

4. The dose recording device according to claim 1, further comprising:
a display screen, exposed from the casing and electrically connected to the circuit board module.

5. The dose recording device according to claim 1, further comprising:
a wireless communication module disposed at the casing and electrically connected to the circuit board module.

6. A dose recording method, adapted for recording an injection information of an injection pen, the injection pen comprising a liquid outlet end, a wheel, a dial gauge linked with the wheel and a press button linked with the wheel, the dose recording method comprising:
executing an optical sensing process, wherein the optical sensing process comprises sensing at least one of a position of the press button of the injection pen and a value on the dial gauge;
executing a vibration sensing process, wherein the vibration sensing process comprises sensing a vibration of the wheel of the injection pen as the wheel rotates;
determining an information of a dose intended to be output by the injection pen according to both an information sensed via the optical sensing process and an information sensed via the vibration sensing process;
when the press button is being pressed, executing a gravity sensing process for determining whether the injection pen is in a work mode or in an air clearing mode, wherein in the work mode, the liquid outlet end of the injection pen is at a lower position in a gravity direction with respect to the wheel, and in the air clearing mode, the liquid outlet end of the injection pen is at an upper position in a gravity direction with respect to the wheel; and
recording the injection information of the injection pen in the work mode, wherein the injection information comprises a value of change of the dial gauge and an injection time.

7. The dose recording method according to claim 6, further comprising:
executing a user's dose confirmation process before the injection pen executes the work mode, wherein the information of the dose intended to be output by the injection pen is output for the user's confirmation.

8. The dose recording method according to claim 6, further comprising:
outputting the injection information after the injection pen executes the work mode.

9. The dose recording method according to claim 6, wherein the optical sensing process further comprises sensing the position of the press button of the injection pen and the value on the dial gauge of the injection pen.

10. The dose recording device according to claim 6, further comprising:
recording a time information of the injection pen in the air clearing mode.
